(19) United States
(12) Patent Application Publication
(10) Pub. No.: US 2008/0112954 A1
(43) Pub. Date: May 15, 2008

(54) Method for treating chronic pain

(75) Inventor: Xue-Jun Song, Irving, TX (US)

Correspondence Address: HOWREY LLP, C/O IP DOCKETING DEPARTMENT, 2941 FAIRVIEW PARK DRIVE, SUITE 200, FALLS CHURCH, VA, 22042-7195, US

(73) Assignee: Parker College of Chiropractic

(21) Appl. No.: 11/983,446

(22) Filed: Nov. 9, 2007

Related U.S. Application Data
(60) Provisional application No. 60/858,252, filed on Nov. 10, 2006.

(51) Int. Cl. A61K 39/395

(52) U.S. Cl. 424/133.1

(57) ABSTRACT

The present invention provides a method for treating a chronic pain by administering to an individual in need of such treatment with a pharmaceutically effective amount of a blocking reagent for ephrinB-EphB signaling. Preferably, the chronic pain comprises a neuropathic pain.

This application claims the benefit under 35 U.S.C. 119(e) of U.S. Provisional Application Ser. No. 60/858,252, filed Nov. 10, 2006, the entire contents of which are incorporated herein by reference.

### BACKGROUND OF THE INVENTION

1. Field of the Invention

The present invention generally relates to medical treatment. Particularly, the present invention relates to a method for treating a chronic pain, more particularly a neuropathic and/or an inflammatory pain, using a blocking reagent for ephrinB-EphB signaling.

2. Description of the Related Art

Chronic pain, such as neuropathic and inflammatory pain, poses a major clinical challenge. Particularly, neuropathic pain is a severe intractable pain, and current drug and non-drug therapies offer substantial pain relief to only no more than half of affected patients. In neuropathic painful conditions, damage to the axons and/or somata of sensory neurons within dorsal root ganglion (DRG) produces painful hyperalgesia accompanied by allodynia (Song et al., 1999, 2003; Zimmermann, 2001). These behavioral effects are associated with spontaneous activity (SA) and hyperexcitability in the affected DRG neurons, atrophic changes and a switch in neurotransmitter phenotype in their central afferent terminals, and alterations in synaptic plasticity, excitatory and inhibitory mechanisms in spinal dorsal horn (DH) neurons (McLachlan et al., 1993; Stucky et al., 2001; Moore et al., 2002; Ji et al., 2003; March et al., 2005; Salter, 2005; Zhang and Xiao, 2005; Zheng and Song, 2005; Song et al., 2006; Zhang and Bao, 2006; Zheng et al., 2007).

Numerous processes have been implicated in neuropathic pain, but the key mechanisms that control its induction and maintenance remain unclear. One possibility is that nerve injury elicits neuronal alterations that recapitulate events during development, including the promotion of synapse formation (Chen et al., 2007). For example, after sciatic nerve injury, noradrenergic perivascular axons sprouted into DRG and formed basket-like structures that could activate axotomized sensory neurons (MacLanchlan et al., 1993). Many studies have also demonstrated switches in the phenotype of neurotransmitters in primary afferent terminals after peripheral nerve injury (MacLanchlan et al., 1993; Ji et al., 2003; Zhang and Xiao, 2005; Zheng and Song, 2005; Moore et al., 2002; Salter, 2005; Chen et al., 2007). Interestingly, Eph receptors and ephrins, which are important in nervous system circuit assembly, continued to be expressed (at lower levels) in the adult central nervous system and, after neural injury, they were upregulated in reactive astrocytes, oligodendrocytes, and neurons (Li et al., 1998; Bundesen et al., 2003; Fitzerald, 2005; Wang and Zhou 2005; Wang et al., 2005; Goldshmit et al., 2006). However, these studies only explored Eph receptors and ephrins in central neuronal development and injury, and did not associate these proteins with any kind of pain, particularly a chronic pain caused by peripheral nerve injury.

A study of Battaglia et al. (2003) suggested that EphB-ephrinB signaling may modulate acute pain processing after peripheral inflammation in the matured nervous system, wherein acute inflammatory pain models of adult rats were used. However, Battaglia's studies did not involve chronic pains, let alone associate ephrinB-EphB signaling with chronic pain, particularly neuropathic pain.

Receptor tyrosine kinases (RTKs) play vital roles in transmitting external signals to the inside of many types of cells. Eph-receptors constitute the largest subfamily of RTKs in the human genome, with 13 members divided into an A-subclass (EphA1-EphA8) and a B-subclass (EphB1-EphB4, EphB6) that have partially overlapping functions. Their ligands, the ephrins, are also divided into two subclasses: ephrinA1-ephrinA5 and ephrinB1-ephrinB3. A-type receptors typically bind to most or all A-type ligands, and B-type receptors bind to most or all B-type ligands (Kullander and Klein, 2002). Both the B-type ephrins and EphB receptors are membrane proteins that initiate bidirectional signaling when the proteins aggregate (Kullander and Klein, 2002; Palmer and Klein, 2007).

Eph RTKs and ephrins are involved in tissue-border formation, cell migration, and axon guidance during development of the nervous system (Krull et al., 1997; Wang and Anderson, 1997; Wilkinson, 2000, 2001). Previous studies found that EphB receptors could also regulate the development of glutamatergic synapses and their plasticity in adult hippocampus by interaction with N-methyl d-aspartate (NMDA) receptors (Dalva et al., 2000; Grunwald et al., 2001, 2004; Takasu et al., 2002; Henderson et al., 2001; Chen et al., 2004), which suggest the possibility that EphB receptor signaling could acutely influence NMDA receptor activity and adult synaptic plasticity in vivo. Battaglia et al. (2003) explored this possibility in acute inflammatory pain models of adult rats and found that EphB receptor was involved in modulating synaptic transmission and acute pain processing at glutamatergic synapses in the dorsal horn (DH) of the spinal cord. However, Battaglia et al. did not explore this possibility in chronic pain conditions, particularly neuropathic pain conditions following peripheral nerve injury.

There is apparently a need for an effective method for treating a chronic pain, particularly a neuropathic pain. The present invention fulfills this long-standing need.

### SUMMARY OF THE INVENTION

The present invention demonstrates contribution of ephrinB-EphB signaling to chronic pains, particularly neuropathic pains, after peripheral nerve injury, and further provides methods for treating chronic pains.

In detail, the present invention is directed to a method for treating a chronic pain by administering to an individual in need of such treatment with a pharmaceutically effective amount of a blocking reagent for ephrinB-EphB signaling. The chronic pain preferably comprises a neuropathic pain.

The foregoing and other advantages of the present invention will be apparent to those skilled in the art, in view of the following detailed description of the preferred embodiment of the present invention, taken in conjunction with the accompanying drawings.

### BRIEF DESCRIPTION OF THE DRAWINGS

The present application file contains at least one drawing executed in color. Copies of this patent application publication with color drawing(s) will be provided by the Office upon request and payment of the necessary fee.

Features of the present invention as well as a preferred mode of use, further objectives, and advantages thereof, will best be understood by reference to the following detailed description of an illustrative embodiment when read in conjunction with the accompanying drawing, wherein:

FIGS. 1A-1H illustrate measurements of thermal (FIGS. 1A, 1C, 1E and 1G) and mechanical (FIGS. 1B, 1D, 1F and 1H) sensitivity of foot withdrawal response in CCI- and sham-operated rats injected (indicated by arrows) with EphB1-Fc, EphB2-Fc, PBS or human Fc in accordance with the present invention.

FIGS. 2A-2B illustrate effects of ephrinB1-Fc, ephrinB2-Fc, PBS or human Fc on thermal hyperalgesia (FIG. 2A) and mechanical allodynia (FIG. 2B) in accordance with the present invention. The arrow indicates the time point of drug injection.

FIGS. 3A-3E illustrate neural responses recorded with whole cell patch electrodes in determining AP threshold (FIG. 3A); neural discharge patterns evoked by depolarizing current (FIG. 3B); and effects of ephrinB1-Fc and EphB1-Fc on AP threshold current, repetitive discharge and the ectopic SA (FIGS. 3C, 3D and 3E, respectively) in accordance with the present invention.

FIGS. 4A-4C illustrate responses of WDR neurons evoked by brush, pressure and pinch applied to the peripheral receptive field from rats that received sham surgery, CCI or CCI plus repeated application of EphB1-Fc (FIG. 4A); responses of each of the WDR neurons tested before and after treatment of EphB1-Fc (FIG. 4B); and spontaneous discharge patterns of WDR neurons recorded in sham-operated and CCI rats (FIG. 4C) in accordance with the present invention.

FIGS. 5A-5H illustrate C-fiber-evoked field potentials recorded before (a) and after (b) tetanic stimulation (indicated by the arrow). LTP training protocol: 100 Hz, 5× threshold current, 0.5 ms, 100 pulses, 4 trains of 1-sec duration at 10-sec intervals (FIGS. 5A-5D); 100 Hz, 5×threshold current, 0.5 ms, 100 pulses, 2 trains of 1-sec duration at 10-sec intervals (FIGS. 5E-5H).

FIGS. 6A-6F illustrate quantification of changes in ephrinB and EphB receptor protein levels after nerve injury. FIGS. 6A and 6D illustrate Western blot results of ephrinB1 and EphB1 receptor protein levels in both the spinal cord and DRG, respectively. FIGS. 6B, 6C, 6E and 6F illustrate quantification of ephrinB1 and EphB1 receptor protein levels in bar graphs.

FIGS. 7A-7F illustrate immunocytochemistry and immunofluorescence staining of ephrinB and EphB receptors in DRG and spinal DH. FIGS. 7A and 7B illustrate presence of ephrinB (green) and EphB receptor (green), respectively, in the DRG cells, which were labeled with propidium iodide (PI, red). FIGS. 7C and 7D illustrate presence of ephrinB (green) and EphB receptor (green), respectively, in cells of the spinal dorsal horn labeled with PI (red).

FIGS. 7E and 7F illustrate presence of ephrinB (green) and EphB receptor (green), respectively, in the spinal dorsal horn with the nociceptive afferent fibers labeled with IB4 (red).

FIGS. 8A-8D illustrate expression of ephrinB and EphB receptors in neurons of the DRG and DH. FIGS. 8A and 8B illustrate presence of ephrinB (green) and EphB (green) receptors, respectively, in DRG neurons (red) from sham-treated and CCI-treated animals. FIG. 8C illustrates absence of ephrinB receptors in IB4 positive terminals in DH neurons from sham-treated and presence of ephrinB receptors (green) in IB4 positive terminals in DH neurons from CCI-treated animals. FIG. 8D illustrates presence of EphB receptors (green) in DH neurons from sham-treated and CCI-treated animals.

### DETAILED DESCRIPTION

In order to provide a clear and consistent understanding of the specification and claims, including the scope given to such terms, the following definitions are provided:

As used herein, the term “chronic pain” refers to a pain that lasts for days to months in mammals.

As used herein, the term “neuropathic pain” refers to a pain that is initiated or caused by a primary lesion or dysfunction in the nervous system.

As used herein, the term “nerve injury-induced neuropathic pain” refers to a peripheral neuropathic pain that occurs when the lesion or dysfunction affects the peripheral nervous system.

As used herein, the term “inflammatory pain” refers to a pain that is caused by inflammation. It can be acute or chronic.

As used herein, the term “hyperalgesia” refers to a pain behavior with an increased response to a stimulus that is normally painful. When the stimulus is heat, the increased response is called “thermal hyperalgesia”.

As used herein, the term “allodynia” refers to a pain behavior caused by a stimulus that does not normally provoke pain. The stimulus could be mechanical (mechanical allodynia) or temperature (cold allodynia).

As used herein, the term “resting membrane potential (RMP)” refers to the electrical potential difference (voltage) across a neuron's plasma membrane.

As used herein, the term “action potential (AP)” refers to a “spike” of positive and negative ionic discharge that travels along the membrane of a neuron. Action potential is an essential feature of animal life.

As used herein, the term “long-term potentiation (LTP)” refers to the long-lasting enhancement in communication between two neurons that results from co-stimulation of the two neurons. For example, pain stimulation can enhance the synaptic plasticity.

As used herein, the term “wide dynamic range (WDR) neuron” refers to the group of neurons in spinal dorsal horn that respond to noxious and non-noxious stimuli applied to the peripheral receptive field and deliver the signals to the higher levels of the nervous system.

As used herein, the term “extracellular recording of single unit” refers to the extracellular, electrophysiological recordings of electric activity from a single neuron in vivo.

As used herein, the term “whole cell patch-clamp recording” refers to a electrophysiological recording technique widely used for recording electrophysiological signals from a single neuron/cell.

As used herein, the term “chronic constriction injury (CCI)” refers to the partial injury to the sciatic nerve as described in the CCI model of Bennett and Xie (1988).

As used herein, the term “chronic compression of dorsal root ganglion (CCD)” refers to a model used to mimic the compressed dorsal root ganglion (DRG) and the nerve roots as described in the CCD model of Hu and Xing (1998) and Song et al. (1999, 2003). In the CCD model, the dorsal root ganglion (DRG) somata are compressed, while the adjacent tissues especially the dorsal roots (central branches of axons of DRG neurons) and the spinal nerve (the peripheral axons of DRG neurons close to the DRG somata) may also be compressed or damaged. It is common in clinic that the disk herniation and/or other diseases or injury would damage these tissues.

As used herein, the term “sham-operated animal” refers to an animal that has received surgery including anesthesia, skin cut and muscles separation, but without any injury to the nerve and/or dorsal root ganglion.

As used herein, the term “sham CCI surgery” refers to the surgical procedure similar to that of CCI, but without damaging the sciatic nerve.

As used herein, the term “sham CCD surgery” refers to the surgical procedure similar to that of CCD, but without compressing the DRG.

As used herein, the term “spontaneous activity (SA)” or “spontaneous discharge” refers to the firing of the DRG neurons without obvious stimulus. The DRG neurons are silent (no spontaneous firing/activity) under normal physiological condition, but become more excitable manifested as spontaneous activity after nerve injury (CCI) and/or DRG compression (CCD).

Bidirectional signaling between ephrins and Eph receptor tyrosine kinases was first found to play important roles during development (e.g., tissue patterning, cell migration and axon guidance), but recently has been implicated in synaptic plasticity in the mature nervous system. The present invention demonstrates that ephrinB-EphB signaling was activated in adult nociceptive neurons following nerve injury in order to promote hyperexcitability of nociceptive sensory neurons, potentiation of their synapses, and consequent central sensitization of spinal DH neurons, hyperalgesia, and allodynia. It is further suggested that ephrinB-EphB receptor signaling was critical for the development of neuropathic pain after peripheral nerve injury or chronic compression of dorsal root ganglion (DRG) (CCD) and for associated changes in the excitability of nociceptive sensory neurons and the strength of their synapses in the spinal cord.

In particular, the present invention demonstrates that intrathecal delivery of blocking reagents for EphB-receptors, e.g., EphB1-Fc and EphB2-Fc chimeras, inhibited the induction and maintenance of nerve injury-induced thermal hyperalgesia and mechanical allodynia. These blockers also prevented other effects of nerve injury, including hyperexcitability of nociceptive DRG neurons, sensitization of spinal dorsal horn (DH) neurons, and long-term potentiation (LTP) of synapses between C fibers and DH neurons. Conversely, an EphB activator, e.g., ephrinB1-Fc, enhanced these effects following nerve injury. In naïve, uninjured animals, intrathecal application of EphB-receptor activators induced thermal hypersensitivity and lowered the threshold for LTP in the DH. Furthermore, Western blot and immunohistochemical analyses showed that ephrinB and EphB receptor proteins were upregulated in the DRG and DH after nerve injury, and at least part of the increased expression occurred in neurons. These results indicate that, by regulating neural excitability and synaptic plasticity at the spinal level, ephrinB-EphB signaling contributes to neuropathic pain.

The present invention further explores therapeutical effects of blocking reagents for ephrinB-EphB signaling in treating chronic pain, particularly a neuropathic pain, after nerve injury.

The present invention is directed to a method for treating a chronic pain by administering to an individual in need of such treatment with a pharmaceutically effective amount of a blocking reagent for ephrinB-EphB signaling.

In a preferred embodiment, the chronic pain comprises a neuropathic pain, which can be caused by peripheral nerve injury or chronic compression of dorsal root ganglion, spinal nerves and/or dorsal roots. More preferably, the chronic pain can further comprise an inflammatory pain that accompanies the neuropathic pain.

The blocking reagent can be an EphB receptor blocker, two examples of which are EphB1-Fc and EphB2-Fc. The blocking reagent can be administrated intrathecally to the individual in need of such treatment in the dosage range of from about 0.5 μg to about 50 μg, preferably, from about 5 μg to about 10 μg. The individual being treated is usually a mammal, preferably, a human.

The following example is given for the purpose of illustrating various embodiments of the invention and is not meant to limit the present invention in any fashion.

### EXAMPLES

### Materials and Methods

Animals and Neuropathic Pain Models

Experimental procedures were performed on adult, male Sprague-Dawley rats (150-200 g) and were conducted in accordance with the regulations of the ethics committee of the International Association for the Study of Pain and approved by the Parker Research Institute Animal Care and Use Committee. Of the 500 rats used in this study, 131 received chronic constriction injury (CCI) of the sciatic nerve, 64 received chronic compression of DRG (CCD), 113 received sham CCI and another 64 with sham CCD surgery, and 128 naïve rats received neither nerve injury nor any surgery. All surgeries were done under anesthesia induced by intraperitoneal injection (i.p.) of sodium pentobarbital (40 mg/kg) and the in vivo recordings were performed under anesthesia by urethane (i.p., 1.5 mg/kg, see below). After surgery, the muscle and skin layers of the rats were sutured.

Peripheral nerve injury was modeled with CCI (Bennett and Xie, 1988; Song et al., 2003). In brief, the left common sciatic nerve of each rat was exposed at the mid-thigh level. Proximal to the sciatic nerve's trifurcation, approximately 7 mm of nerve was freed of adhering tissue and four ligatures (4-0 chronic gut) were tied loosely around it with about 1 mm between ligatures.

Chronic compression of DRG (CCD) was produced by surgically implanting stainless steel rods unilaterally into the intervertebral foramen at L₄ and L₅ using the procedure for CCD previously described (Song et al., 1999, 2003). In brief, the rats (n=8) were anesthetized; paraspinal muscles were separated from the mammillary and transverse processes, and the intervertebral foramina of L₄ and L₅ were exposed. One stainless steel L-shaped rod, 4×2 mm in length and 0.6 mm in diameter, was implanted into the foramen at L₄ and another at L₅.

Assessment of Thermal Hyperalgesia and Mechanical Allodynia

Thermal hyperalgesia was assessed by measuring foot withdrawal latency to heat stimulation using a protocol previously described (Song et al., 2003, 2006). Each rat was placed in a box (22×12×12 cm for rat) containing a smooth glass floor. The temperature of the glass was measured and maintained at 26-26.5° C. A heat source (IITC Model 336 Analgesia Meter, Series 8, available from Life Science, Woodland Hills, Calif.) was focused on a portion of the hindpaw, which was flush against the glass, and a radiant thermal stimulus was delivered to that site. The stimulus shut off automatically when the hindpaw moved (or after 20 sec to prevent tissue damage). The intensity of the heat stimulus was maintained constant throughout all experiments. The elicited paw movement occurred at latency between 9 and 14 sec in control animals. Thermal stimuli were delivered 4 times to each hind paw at 5-6 min intervals.

Mechanical allodynia was determined by measuring incidence of foot withdrawal in response to mechanical indentation of the plantar surface of each hindpaw with a sharp, cylindrical probe, using a protocol we described previously (Song et al., 1999, 2003, 2006). Von Frey filaments capable of exerting forces of 10, 20, 40, 60, 80 and 120 mN with a uniform tip diameter of 0.1 mm, were applied to 10 designated loci distributed over the plantar surface of the foot. The filaments were applied in order of ascending force, and each filament was applied alternately to each foot and to each locus. The duration of each stimulus was approximately 1 sec and the inter-stimulus interval was approximately 10-15 sec. The incidence of foot withdrawal was expressed as a percentage of the 10 applications of each stimulus and the percentage of withdrawals was then plotted as a function of force. The von Frey withdrawal threshold was defined as the force that evoked a minimum detectable withdrawal observed on 50% of the tests given at the same force level. For cases in which none of the specific filaments used evoked withdrawals on exactly 50% of the tests, linear interpolation was used to define the threshold.

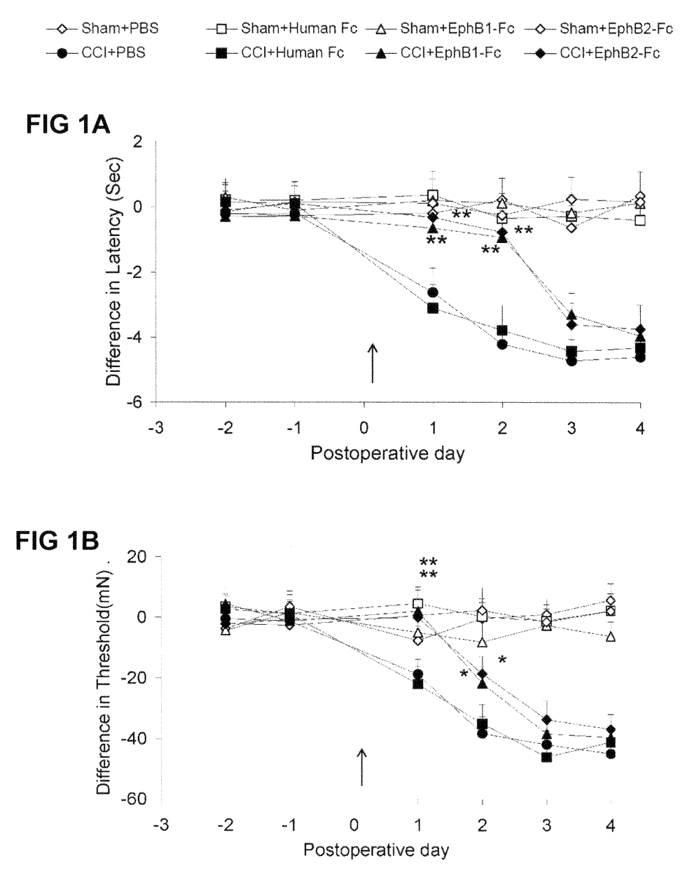

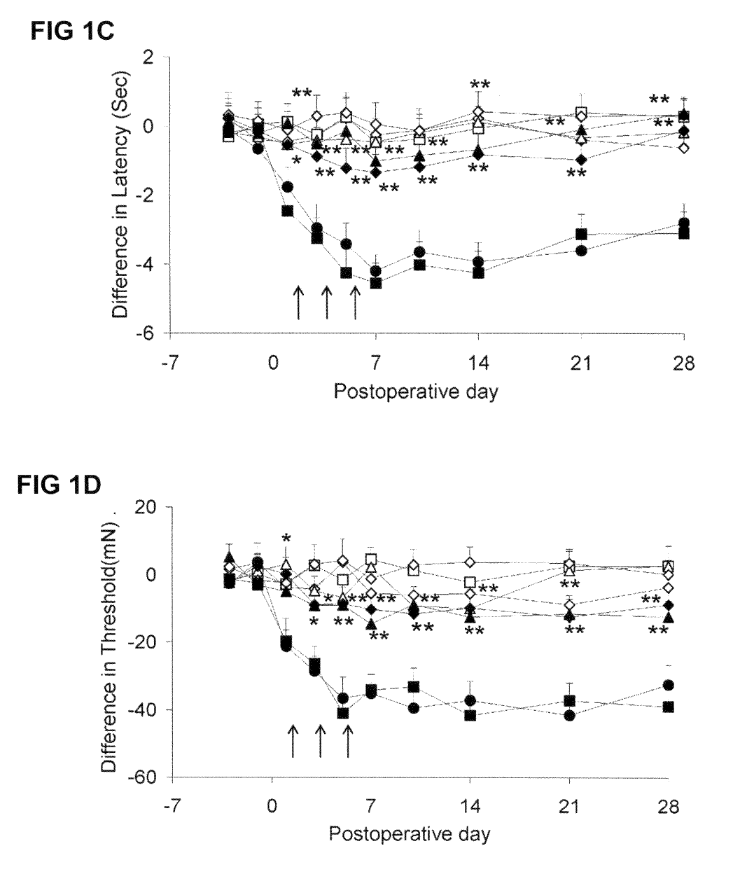

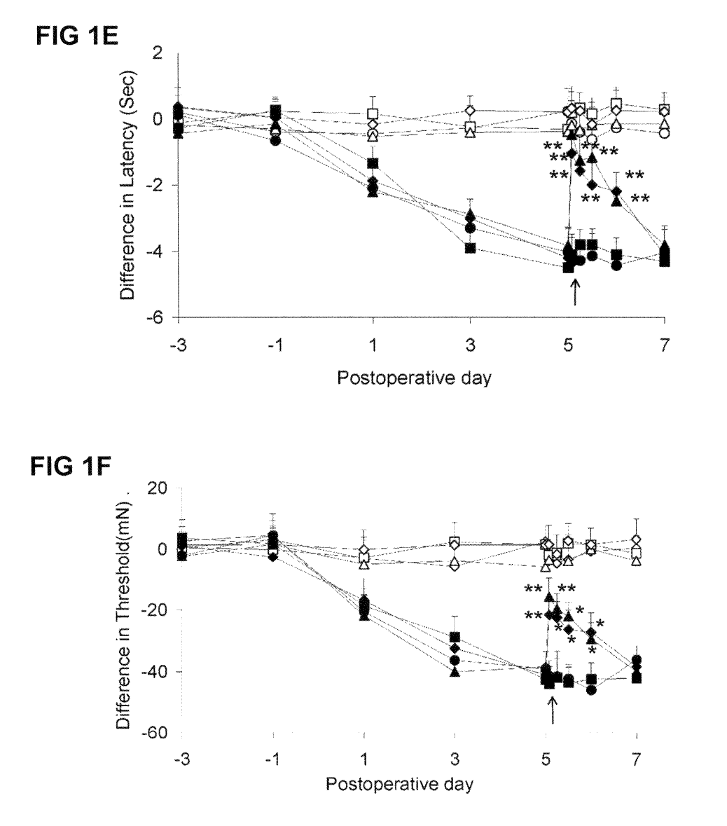

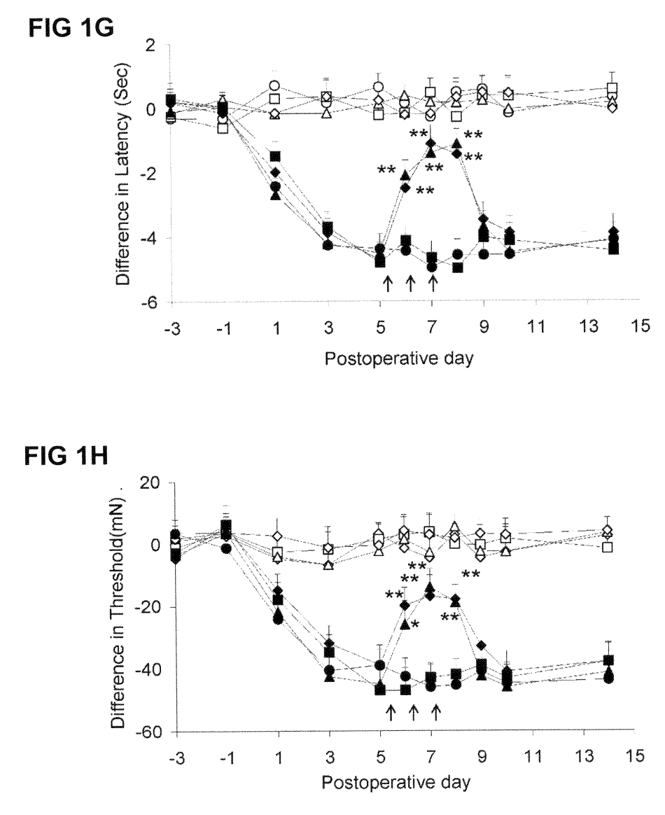

To reduce preexisting differences in responsiveness among individuals, withdrawal latencies or threshold were normalized, respectively, by subtracting each value on the treated side from the corresponding value on the contralateral side and expressing the results as difference scores (FIGS. 1A-H). These figures illustrate repeated measurements of thermal and mechanical sensitivity of the foot withdrawal response in CCI- and sham-operated rats injected (i.t.) with EphB receptor blocking reagents (e.g., EphB1-Fc and EphB2-Fc), or PBS or human Fc. Each group included 8 rats, which received both thermal and mechanical tests. Separate groups of rats were used in the groups with different treatments. Arrows indicate the time point of each injection. *p<0.05 and **p<0.01 indicate significant differences between groups of CCI and CCI treated with EphB receptor blockers (2-way ANOVA with repeated measures followed by Bonferroni post hoc tests).

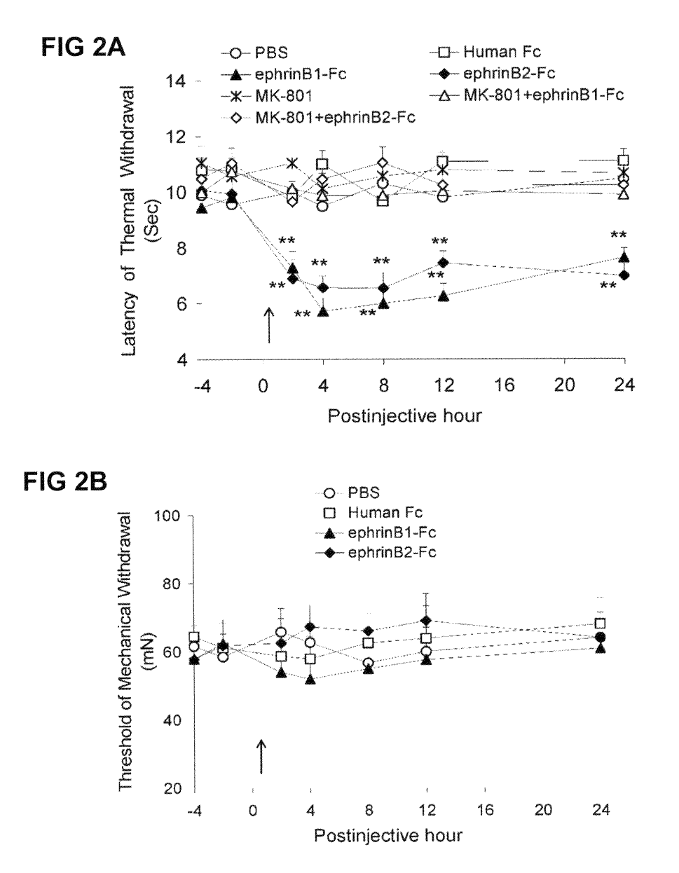

For the results expressing intrathecal ephrinB1/Fc-induced hyperalgesia, the values are mean values of both feet (FIGS. 2A and 2B). 2 μg of EphrinB1-Fc or ephrinB2-Fc, human Fc or PBS was injected (i.t.) to the rats. Each group included 8 rats, which received both thermal and mechanical tests. The arrow indicates drug injection (i.t.) at the time point. *p<0.05 and **p<0.01 indicate significant differences between groups of the treatment and the PBS control (2-way ANOVA with repeated measures followed by Bonferroni post hoc tests).

Excised, Intact Ganglion Preparation

DRG neurons were tested while still in place in excised ganglia. These intact DRG neurons display excitability properties that are more normal than those of dissociated DRG neurons (Song et al., 2003, 2006; Zheng et al., 2007). The protocols were described previously (Song et al., 2006). In brief, under anesthesia, the sciatic nerve was isolated and transected at the mid-thigh level, and its proximal portion traced to the ganglia. After a laminectomy was performed, ice-cold, oxygenated, buffered solution containing 140 mM NaCl, 3.5 mM KCl, 1.5 mM CaCl₂, 1 mM MgCl₂, 4.5 mM HEPES, 5.5 mM HEPES-Na and 10 mM glucose (pH 7.3, osmolarity 310-320 mOsm) was dripped onto the surface of the ganglion during the procedure. The ganglia from the left side of the L₄ and L₅ segments were removed and placed in 35-mm petri dishes containing the ice-cold, oxygenated, buffered solution. The perineurium and epineurium were peeled off and the attached sciatic nerve and dorsal roots transected adjacent to the ganglion. The otherwise intact ganglion was then treated with collagenase (Boehringer type P, 1 mg/ml, Ridgefield, Conn.) for 30 min at 35° C., transferred to the recording chamber, and mounted on the stage of an upright microscope (BX50-WI, Olympus, Japan). A U-shaped stainless steel rod with 4 crossing silver wires held the ganglion gently in place. The DRG was incubated in the oxygenated, buffered solution at room temperature.

Whole Cell Patch-Clamp Recordings

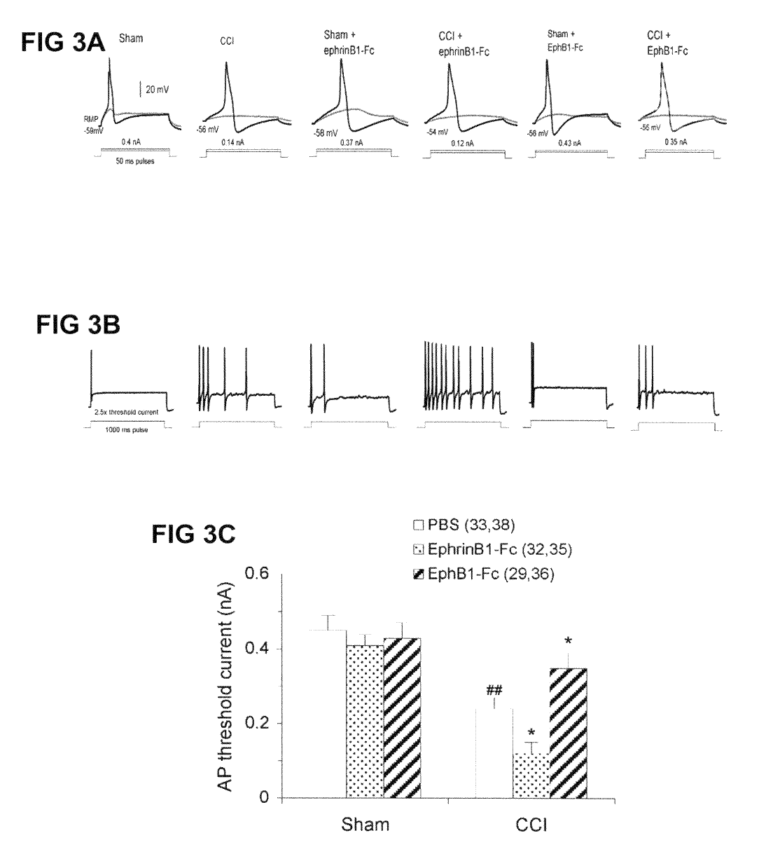

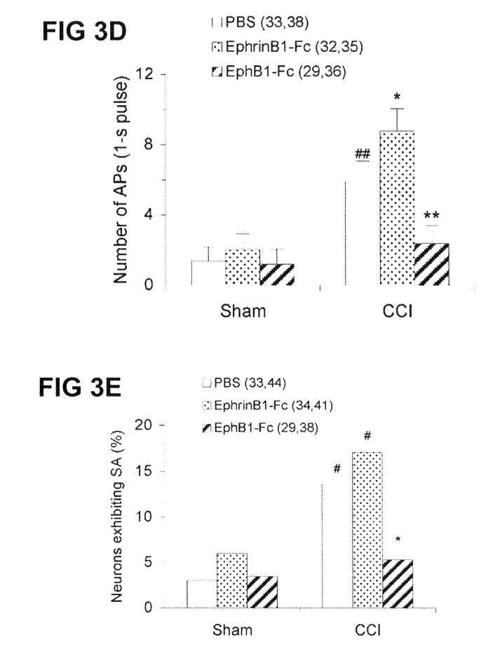

Whole cell patch-clamp recordings were made from small neurons in the intact DRG (soma diameter 15-30 μm; membrane input capacitance ≦45 pF). Glass electrodes were fabricated with a Flaming/Brown micropipette puller (P-97, available from Sutter Instruments, Novato, Calif.). Electrode impedance was 3-5 MΩ when filled with saline containing 120 mM K⁺-gluconate, 20 mM KCl, 1 mM CaCl₂, 2 mM MgCl₂, 11 mM ethyleneglycol-bis-(β-aminoethyl-ether) N,N,N′,N′,-tetraacetic acid (EGTA), 2 mM Mg-ATP and 10 mM HEPES-K (pH 7.2, osmolarity 290-300 mOsm). Electrode position was controlled by a 3-D hydraulic micromanipulator (MHW-3, available from Narishige, Japan). When the electrode tip touched the cell membrane, gentle suction was applied to form a tight seal (serial resistance >2 GΩ). Under −70 mV command voltage, additional suction was applied to rupture the cell membrane. After obtaining the whole cell mode, the recording was switched to bridge mode (I=0) and the resting membrane potential (RMP) was recorded. During the procedure, action potential (AP) current threshold, repetitive discharge evoked by depolarizing current, ectopic spontaneous activity (SA) and other electrophysiological properties of DRG neurons were monitored (FIGS. 3A-3E).

Neural responses were recorded with whole cell patch electrodes under current clamp during the test sequence used to determine AP threshold. Only two of the depolarizing 50-ms pulses (bottom) and corresponding responses (top) were illustrated in each case (FIG. 3A). FIG. 3B illustrates neural discharge patterns evoked by depolarizing current; and FIGS. 3C-3E illustrate effects of EphB receptor activator ephrinB1-Fc and blocker EphB 1-Fc on AP threshold current, repetitive discharge and the ectopic SA, respectively. *p<0.05 and **p<0.01 indicate significant differences compared with the PBS-treated corresponding group (sham or CCI). ^{#}p<0.05 and ^{##}p<0.01 indicate the significant differences compared with the PBS-treated sham group. “EphB1-Fc in vivo” indicates that the neurons were from CCI rats previously given repeated injections of EphB1-Fc, which did not exhibit hyperalgesia on the day of electrophysiological recording. The numbers of cells tested in each group are shown in parentheses. Application of all drugs (ephrinB1-Fc, EphB1-Fc all in 5 μg/100) began 30 min prior to and continued during the 3-4 hr of electrophysiological recording.

Extracellular Recordings of Single Units and LTP of Dorsal Horn Neurons In Vivo

Under anesthesia, a cannula was inserted into the trachea to allow artificial ventilation and another cannula containing heparin (0.03%) saline was inserted into the left carotid artery to monitor the blood pressure. The rat was placed in a stereotaxic frame, then was paralyzed by an intravenous injection of pancuronium bromide (4 mg/kg) and artificially ventilated with air at a tidal volume of 15 ml/kg. Adequate anesthesia was confirmed intermittently during neuromuscular blockade in terms of the following two criteria: (1) the pupils were constricted; (2) the blood pressure remained stable during noxious stimulation. Core body temperature was monitored through a thermister probe inserted into the rectum and maintained at 37.5±0.5° C. by means of a feedback-controlled heating pad under the ventral surface of the abdomen. In each experiment phosphate-buffered saline (PBS) was intermittently administered via a jugular vein cannula to maintain hydroelectrolyte balance. A laminectomy was performed to expose the lumbar spinal cord. The dura mater was then removed the exposed cord was immediately covered with warm agar (2% in saline). After the agar hardened, a small hole was made above the recording site for application of drug or vehicle. At the end of each experiment, the animals were killed by an overdose of intravenous pentobarbital (200 mg/kg).

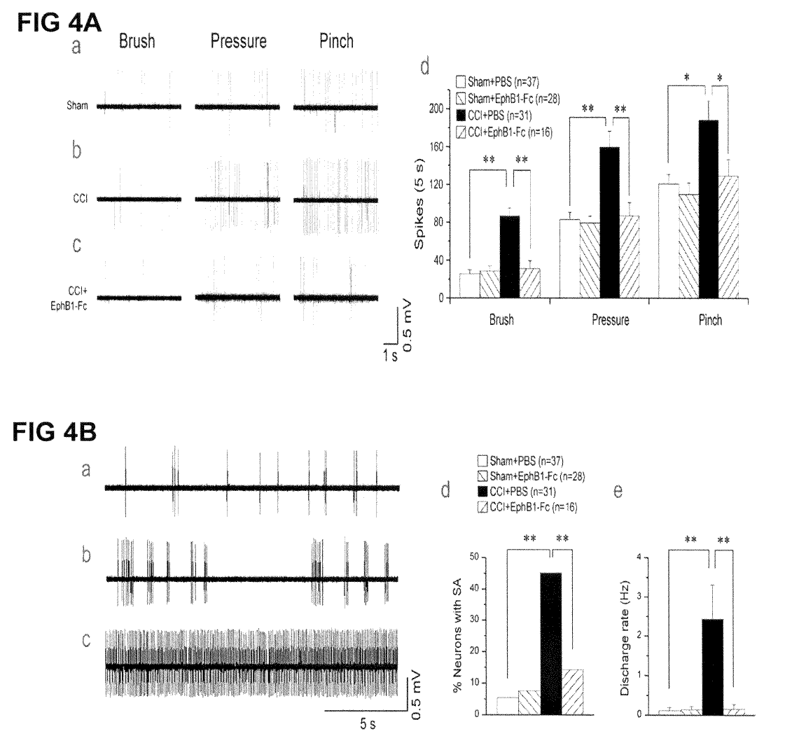

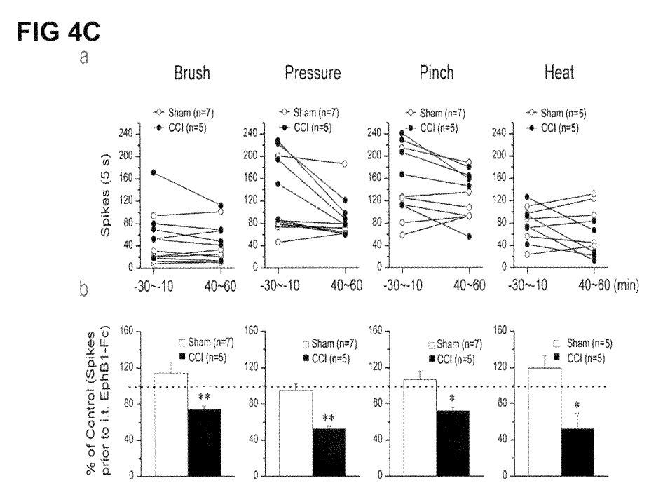

In vivo extracellular recordings of single units were performed on rats 7-14 days after sham surgery, nerve injury or EphB1-Fc treatment. The recordings were made at a depth of 100-500 μm from the surface of the spinal cord in lumbar enlargement at L₄₋₅ with glass capillary microelectrodes (DC resistances 8-12 MΩ filled with 2 M NaCl). Explorations with microelectrodes were made in DH using an electronically controlled microstepping manipulator. Light stroking and probing of the skin at the ipsilateral hindpaw was used as a search stimulus to identify a DH neuron. The DH neuron was identified as wide dynamic range (WDR) neurons if (1) having a receptive field consisting of a small low threshold center and a large high threshold surround; (2) responding with an increasing firing rate to brush, pressure and noxious pinch applied to the low threshold center, while there was only a response to noxious pinch applied to the high threshold surround; (3) showing no evident adaptation when continuous stimulation was applied to the low threshold center. After identification of a single WDR unit, the spontaneous activity was monitored and recorded (if any) for 20 seconds. The quantitative response to mechanical stimulation was evaluated by applying light brush (brushing the skin with a cotton swapper), innocuous pressure (by a constant-force forceps; force of grip 120 g/mm²) and noxious pinch (by similar forceps but with much smaller contact area; 550 g/mm²) to the WDR neuronal receptive field low threshold center. The thermal response was obtained by immersing the hindpaw into 47° C. water bath. All the stimuli were applied for 5 sec and separated from each other by at least 30 min to avoid sensitization of WDR neurons and damage to the receptive fields (FIGS. 4A-4C).

Examples of responses of WDR neurons evoked by brush, pressure and pinch applied to the peripheral receptive field from rats that received sham surgery, CCI or CCI plus repeated application of EphB1-Fc were given in a, b and c respectively, and data summarized in d of FIG. 4A. The numbers of WDR neurons tested in each group are shown in parentheses. Responses of each of the WDR neurons tested before and after treatment of EphB1-Fc (10 μg) were shown in a, and summarized in b of FIG. 4C. In this experiment, each rat only received one application of EphB1-Fc. Examples of spontaneous discharge patterns of WDR neurons recorded in CCI rats were shown in a, b and c, and data summarized in d and e of FIG. 4B. *p<0.05; **p<0.01.

Long-term potentiation (LTP) tests were performed on naïve rats. The protocol for the electrophysiological recordings of C-fiber evoked field potentials was similar to that described previously (Liu and Sandkuhler, 1998). The recordings were made at depths of 200-500 μm from the surface of the spinal cord between L₄-L₅ with glass capillary microelectrodes (DC resistances 3-5 MΩ filled with 2 M NaCl). The sciatic nerve was stimulated by a bipolar platinum hook electrode. Single square pulses (0.5 ms duration) were delivered every 5 min to the sciatic nerve were used as test stimuli. The strength of stimulation was adjusted to 2 times the threshold for a C-fiber response. LTP was induced by tetanic stimulation consisting of 100 electrical pulses, each 0.5 ms, 100 Hz, at 5× the threshold current of C-fibers, given in 4 trains or, when subthreshold, in 2 trains of 1-sec duration at 10-sec intervals to the sciatic nerve (FIGS. A-H). Examples of the C-fiber-evoked field potentials given in each figure were recorded before (a) and after (b) tetanic stimulation (indicated by the arrow therein) or ephrinB1-Fc.

Western Blot

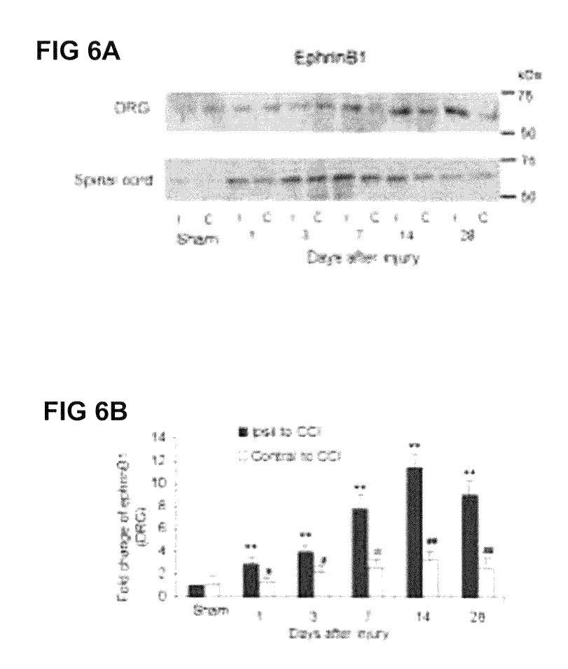

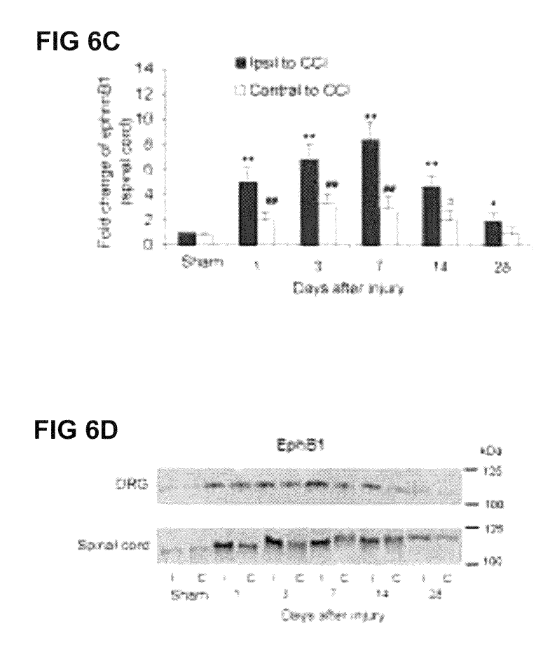

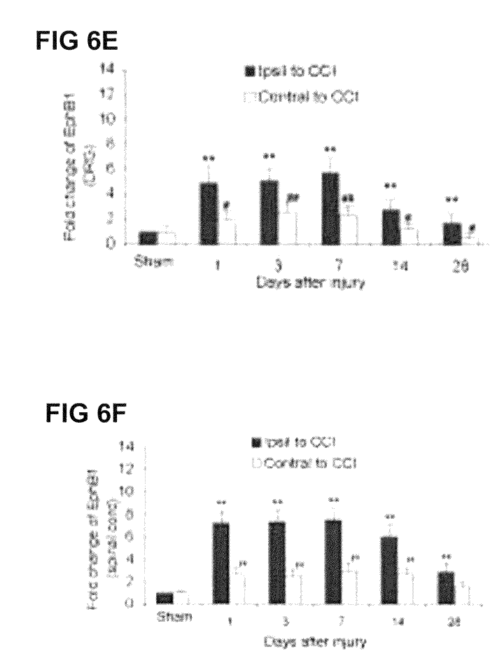

The DRGs (L₄ and L₅) and corresponding spinal cord segments (L₃-L₆) were quickly extracted and immediately frozen in liquid nitrogen, and stored at −80° C. Tissue samples were homogenized in lysis buffer (pH 7.4): 20.0 mM Tris-HCl, 150.0 mM sodium chloride, 50.0 mM NaF, 1.0 mM sodium orthovanadate (Na₃VO₄), 1% Triton X-100 (TX-100), 10% glycerol, 0.1% SDS, 2.0 mM phenylmethylsulfonyl fluoride (PMSF), 1.0 mM dithiothreitol (DTT), and protease inhibitors cocktail 0.02% (v/v). The homogenates were centrifuged at 800 g for 15 min at 4° C. to delete debris. Protein concentrations were determined by Bradford assay (Bradford, 1976). Protein samples (30 μg per lane) were separated using 7.5% and 10% SDS-polyacrylamide gel electrophoresis and transferred onto nitrocellulose membrane. The gels were stained with Coomassie blue to confirm equal amounts of protein loaded in each lane. The membranes were incubated overnight at 4° C. with the following primary antibodies: EphrinB1 (H-70, sc-20723) (1:400), a rabbit polyclonal antibody raised against amino acids 171-240 mapping near the C-terminus of ephrinB1 of human origin; EphB1 (H-80, sc-28979) (1:400), a rabbit polyclonal antibody raised against amino acids 251-730 mapping within an N-terminal extracellular domain of EphB1 of human origin (both antibodies obtained from Santa Cruz Biotechnology, Inc., Santa Cruz, Calif., and recommended for detection of ephrinB1 and EphB1 of mouse, rat and human origin by Western blotting). The membranes were extensively washed with Tris-buffered saline tween-20 and incubated for 1 hr with the secondary antibody conjugated with alkaline phosphatase (1:1000) at room temperature. The immune complexes were detected by using a NBT/BCIP assay kit. The scanned images were imported into ImageJ software (NIH, USA) (FIGS. 6A-6F). Scanning densitometry was used for semiquantitative analysis.

Western blot results illustrating temporal changes of ephrin-B1 and EphB1 receptor protein levels in the spinal cord and DRG are shown in FIGS. 6A and 6D, respectively. The L₄-L₅ segments of spinal cord from each rat were separated into two parts, i.e., ipsilateral (I) and contralateral (C) to the injured nerve, and analyzed separately. Each sample of DRG consisted of L₄ and L₅ ganglia from each rat. Quantifications of ephrin-B1 and EphB1 receptor protein levels in bar graphs are shown in FIGS. 6B, 6C, 6E and 6F. Fold changes were standardized by protein level in the corresponding group of sham ipsilateral (set as 1). Four samples were used at each time point presented, with each sample consisted of 2 DRGs (L₄ and L₅ of I or C), half spinal cord (I and C) from one rat. *p<0.05; **p<0.01 indicate significant differences compared with that ipsilateral to surgery in sham group. #p<0.05; ##p<0.01 indicate significant differences compared with that ipsilateral to CCI in the same group.

Immunocytochemistry and Immunofluorescence Staining

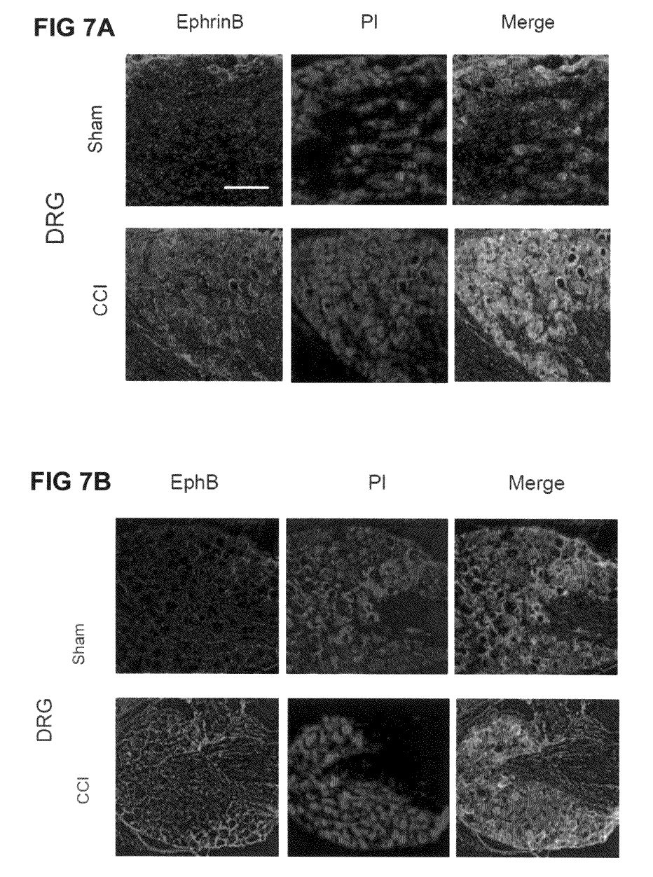

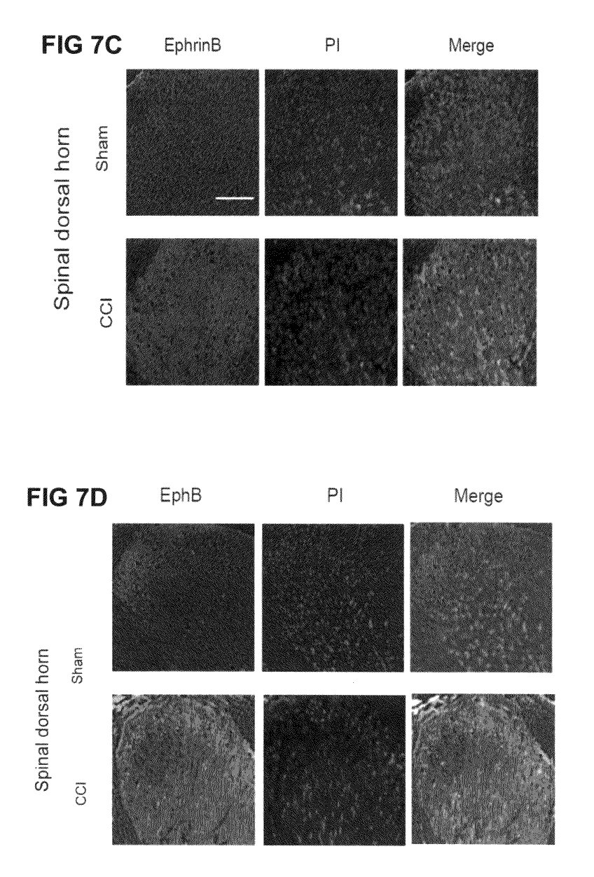

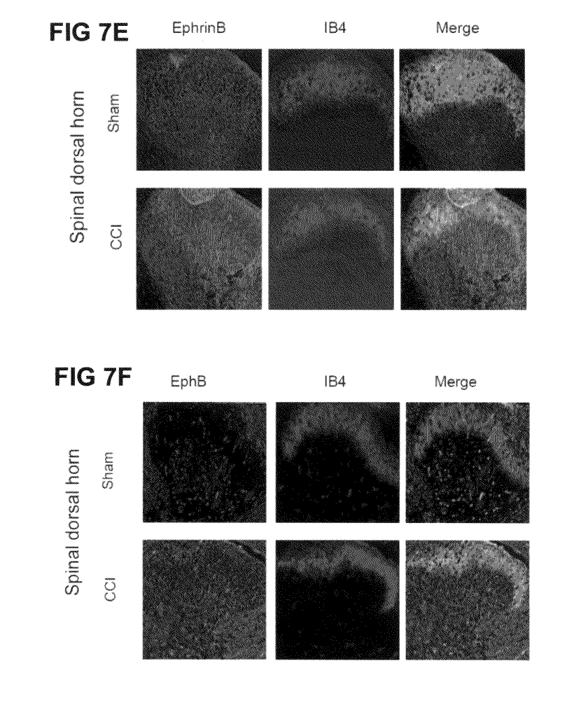

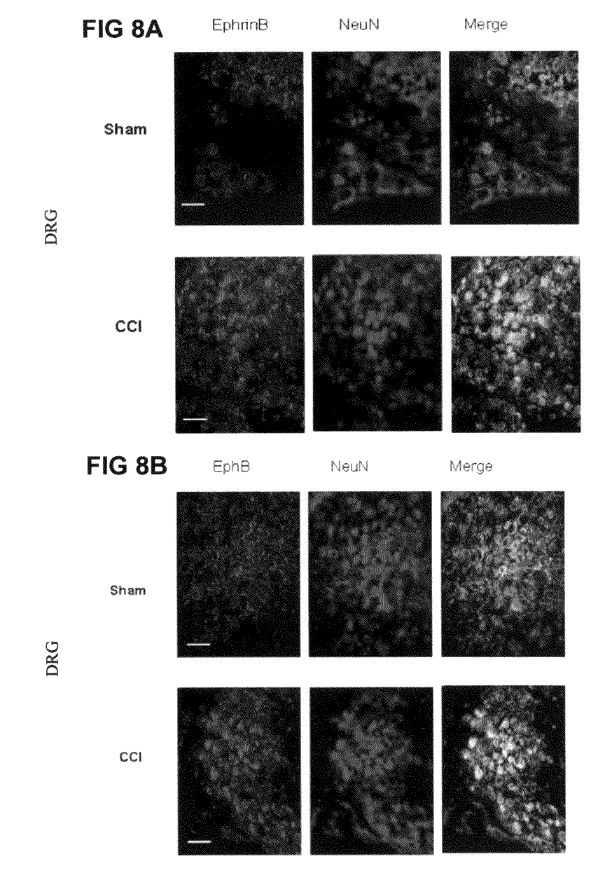

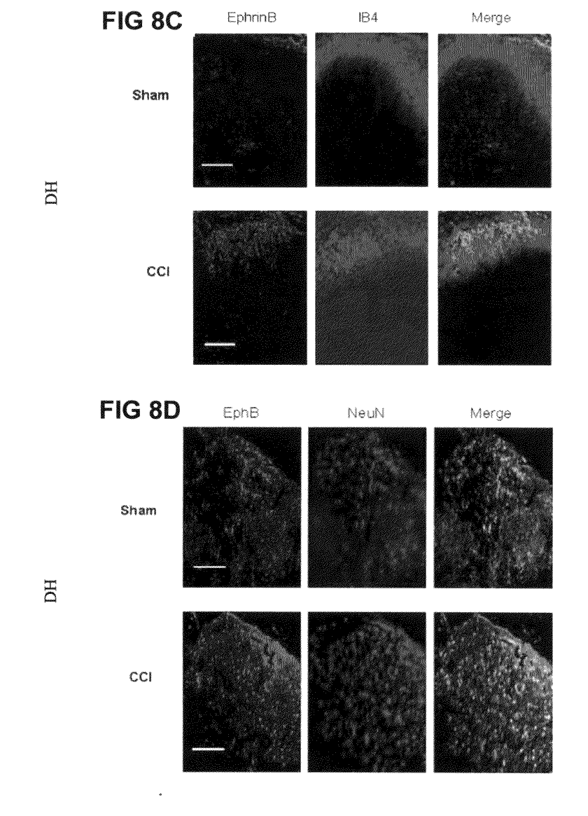

Under anesthesia, rats were perfused transcardially with cold saline 100 ml, followed by 350 ml of ice-cold 4% paraformaldehyde solution. The DRGs (L₄ and L₅) and corresponding spinal cord (L₃-L₆) were dissected out and post-fixed at 4° C. for 4 hr with paraformaldehyde, followed by overnight cryoprotection at 4° C. in 30% sucrose. Tissue sections were embedded in O.C.T. (an abbreviation for “Optimum Cutting Time”) compound (sold under TISSUE TEK II™ by Miles Scientific, a Division of Miles Laboratories, Pittsburgh, Pa.). Coronal sections (10 μm) were cut using a Leica CM1850 cryostat (Leica Microsystems, Germany) and kept in PBS. The sections were rinsed in 10% methanol and 0.03% H₂O₂ in 0.1 M PBS for 30 min and then blocked in 3% normal goat serum (NGS) with 0.2% Triton X-100 (NGST) two times for 10 min each, and then incubated with rabbit polyclonal anti-EphB1 antibody (1:200), rabbit polyclonal anti-ephrinB1 (1:200) (both obtained from Santa Cruz Biotechnology, Inc., Santa Cruz, Calif.). Rabbit IgG was used as an isotype control. All antibodies were diluted in 3% NGST before adding to tissue sections. After overnight incubation with primary antibody at 4° C., tissue sections were washed and incubated at room temperature in a dark humidity chamber with secondary antibody (1:200) (fluorescent labeled anti rabbit IgG, (Vector Laboratories, Inc., Burlingame, Calif.) for 1 hr. After a further PBS wash, the slides were incubated with propidium iodideiodide (PI) counterstaining solution for 5 minutes. Part of the sections were incubated with Alexa Fluor-594 conjugated to IB4 (Molecular Probes, Dilution 2 μg/ml) for 2 hr at room temperature. Some sections were incubated with mouse anti-neuronal nuclear protein (NeuN) at 4° C. for 24 h to identify neurons (1:100; Chemicon, Billerica, Mass.). All antibodies were diluted in 3% NGST. The sections were then washed 3 times with PBS to remove excess antibodies. The primary antibodies were detected with goat anti-rabbit or anti-mouse IgG tagged with fluorescein or rhodamine. Once washed and dried, the slides were mounted using Vectashield H-1400 (Vector Laboratories, Inc., Burlingame, Calif.) as mounting medium. Images were collected with an immunofluorescence microscope (BX51WI, CCD-ODP70, Olympus, Japan) (FIGS. 7A-7F; FIGS. 8A-8D) (scale bars: 100 μm in DRG (FIGS. 7A and 7B; FIGS. 8A and 8B); 200 μm in the spinal dorsal horn (FIGS. 7C-7F; FIGS. 8C and 8D). Magnifications: 100× for all the images).

Drug Application

Contributions of ephrinB-EphB signaling to the development and maintenance of hyperalgesia, hyperexcitability of DRG neurons and hyperexcitability and enhanced synaptic plasticity of DH neurons were investigated by applying EphB receptor activators or blocking reagents in vivo or in vitro. In the behavioral studies, in vivo application (i.t., in 20 μl PBS) of the EphB receptor activator ephrinB1-Fc chimera (2 or 5 μg) (this molecule binds to EphB 1-4 and EphA3; mouse recombinant; Sigma, E 0653) and ephrinB2-Fc chimera (2 or 5 μg) (this molecule binds to EphB1-4 and EphA4; mouse recombinant; Sigma, E 0778), EphB receptor blocking reagents EphB1-Fc chimera (5 or 10 μg) (this molecule binds to ephrinB1-3 and ephrinA1-4; rat recombinant; Sigma, E9277) and EphB2-Fc chimera (5 or 10 μg) (this molecule binds to ephrinB1-3; mouse recombinant; Sigma, E9402) and the human IgG Fc fragment (Jackson, Fc control) were administrated intrathecally by means of lumbar puncture under brief inhalational anesthesia. In the electrophysiological studies of DH neurons, the above drugs and/or NMDA receptor antagonist MK-801 (20 μg, Sigma) were topically applied to the small hole previously made in the agar above the recording site. In the in vitro studies of DRG neurons, the drugs were added into the bath to excised DRG with the buffered solution: ephrinB1-Fc, 2 or 5 μg/100 μl; EphB1-Fc, 5 μg/100 μl.

Statistical Tests

Changes in withdrawal latencies over time were tested with two-way ANOVA with repeated measures followed by Bonferroni post hoc tests. Individual t-tests were used to test specific hypotheses about differences between each operated or drug-treated group and its corresponding control group for each electrophysiological parameter tested. x² tests were used to identify differences in the incidence of effects. All data are presented as mean ±SE. Statistical results are considered significant if p<0.05.

Results

Blocking EphB Receptor Activation Inhibits Induction and Maintenance of Thermal Hyperalgesia and Mechanical Allodynia by Nerve Injury or DRG Compression

Peripheral nerve injury mimicked by chronic constriction injury (CCI) of the sciatic nerve produced a rapid onset and long-lasting thermal hyperalgesia and mechanical allodynia. The present study showed that intrathecal administration (i.t.) of reagents that block the activation of EphB1 receptors, the chimeric molecules of EphB1-Fc and EphB2-Fc, prevented the development of thermal hyperalgesia and mechanical allodynia after nerve injury. A single injection of 10 μg EphB1-Fc or EphB2-Fc 30 min prior to injury delayed the onset of thermal hyperalgesia and mechanical allodynia for 2 days (FIGS. 1A and 1B). Repeated daily injections of EphB1-Fc or EphB2-Fc for 3 days, starting 30 min prior to injury, significantly inhibited thermal hyperalgesia and mechanical allodynia for at least 28 days postoperative (FIGS. 1C and 1D).

The present study also showed that previously induced thermal hyperalgesia and mechanical allodynia were also significantly inhibited by a single injection of EphB1-Fc or EphB2-Fc, but the pain returned within 24 hours (FIGS. 1E and 1F). Following nerve injury, repeated injection of EphB1-Fc or EphB2-Fc also produced transient inhibition, with the pain returning within 24 hours after the last injection (FIGS. 1G and 1H). No significant effects of these treatments were observed on thermal or mechanical thresholds in the feet contralateral to the injury or in sham-operated animals. Injections of PBS or human Fc did not significantly alter the thermal and/or mechanical thresholds in the CCI or sham-operated rats tested (FIGS. 1A-1H).

Effects of two reagents, ephrinB1-Fc and ephrinB2-Fc, known to activate EphB receptors were also examined. A single injection (2 μg, i.t.) of either ephrinB1-Fc or ephrinB2-Fc into a naïve animal caused a short-latency, prolonged thermal hypersensitivity. Injection of ephrinB1-Fc, however, did not induce obvious mechanical allodynia in any of the rats tested. Thermal hypersensitivity induced by injection of ephrinB1-Fc or ephrinB2-Fc was prevented by prior injection of an NMDA receptor antagonist, MK-801 (or dizocilpine) (20 μg). Injection of human Fc did not induce any hypersensitivity (FIGS. 2A and 2B). These findings were consistent to those reported previously (Battaglia et al., 2003).

Currently available reagents do not distinguish among different ephrinB and EphB receptor types. However, the lack of effect of human Fc on the behavioral responses of sham-treated and naïve animals excluded a non-specific hyperalgesic effect of the Fc portion of the molecule. These results indicate that activation of ephrinB-EphB signaling in the spinal cord and/or DRG plays important roles in development of the neuropathic pain.

The EphB blockers produced similar inhibitory effects on the thermal hyperalgesia and mechanical allodynia either elicited in CCI model or induced by DRG compression. The following studies were focused on the effects of ephrinB-EphB receptor signaling in the CCI model of neuropathic pain.

Blocking EphB Receptor Activation Prevents Hyperexcitability of DRG Neurons after Nerve Injury

Hyperexcitability of DRG neurons following nerve injury contributes to the sensitization of central nociceptive neurons in the dorsal horn (DH), leading to chronic pain and hyperalgesia. The present study examined whether ephrinB-EphB signaling contributed to nerve injury-induced hyperexcitability of the nociceptive sensory neurons in vitro in intact DRG using patch-clamp whole cell recordings. Hyperexcitability of DRG neurons after CCI and other forms of injury is often manifested as a decrease in action potential (AP) current threshold, increased repetitive discharge, and spontaneous activity (SA) (e.g., Bennett and Xie, 1988; Study and Kral, 1996; Abdulla and Smith, 2001; Song et al., 1999, 2003, 2006; Zheng et al., 2007). These three electrophysiological properties were evaluated to test the possibility that ephrinB-EphB signaling contributed to the hyperexcitability of small DRG neurons produced by CCI (FIG. 3). Bath application (or perfusion) of the EphB receptor blocking reagent, EphB1-Fc (5 μg/100 μl), onto the ganglion following CCI significantly reduced the hyperexcitability of small DRG neurons. Furthermore, DRG neurons taken on postoperative 7-14 days from animals that had received repeated treatments of EphB1-Fc on days 0-2 (10 μg, 3 doses, i.t.) exhibited no pain and were not hyperexcitable. On the other hand, bath application of the EphB receptor activator, ephrinB1-Fc (5 μg/100 μl), further enhanced hyperexcitability of CCI-DRG neurons, but did not significantly alter excitability of sham-operated DRG neurons. These results indicate that activation of EphB receptors is required for hyperexcitability of DRG neurons induced by CCI.

Blocking EphB Receptor Activation Prevents Sensitization of DH Neurons after Nerve Injury

Nerve injury is known to enhance responses of DH neurons to innocuous and noxious stimuli applied to their peripheral receptive field (Hains et al., 2004). Sensitization of nociceptive and wide dynamic range (WDR) neurons in the DH may directly contribute to behavioral painful syndromes. The present study investigated roles of EphB receptors in sensitization of WDR neurons in vivo, 7-14 days after CCI. The results showed that responses of WDR neurons to innocuous brush and pressure and painful pinch applied to the paw significantly increased after CCI. Such enhanced responses were greatly inhibited in the animals that received repeated treatments of EphB1-Fc on days 0-2 (10 μg, i.t., 3 doses) (FIG. 4A). Topical application of EphB1-Fc (10 μg, i.t.) onto the spinal cord after baseline recording during the experiment also significantly reduced the enhanced responses of WDR neurons to the innocuous and noxious stimulation following CCI, but had no effect on the neural responses in sham-operated animals (FIG. 4B). In addition, nerve injury significantly increased the percentage of WDR neurons exhibiting spontaneous activity (SA) (CCI vs. sham: 42%, n=31 vs. 5.4%, n=37,p<0.01) and caused a significantly higher spontaneous discharge rate (2.4±0.9 Hz vs. 0.15±0.1 Hz, p<0.01). This increased SA was significantly reduced by repeated injection of EphB1-Fc (FIG. 4C). These results indicate that activation of EphB receptors is required for sensitization of WDR neurons after nerve injury.

EphrinB-EphB Signaling Contributes to LTP in Synapses between C-fibers and DH Neurons

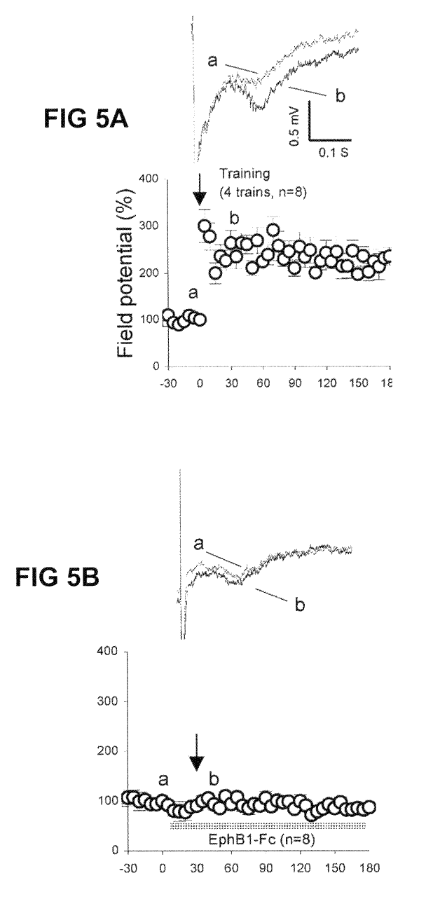

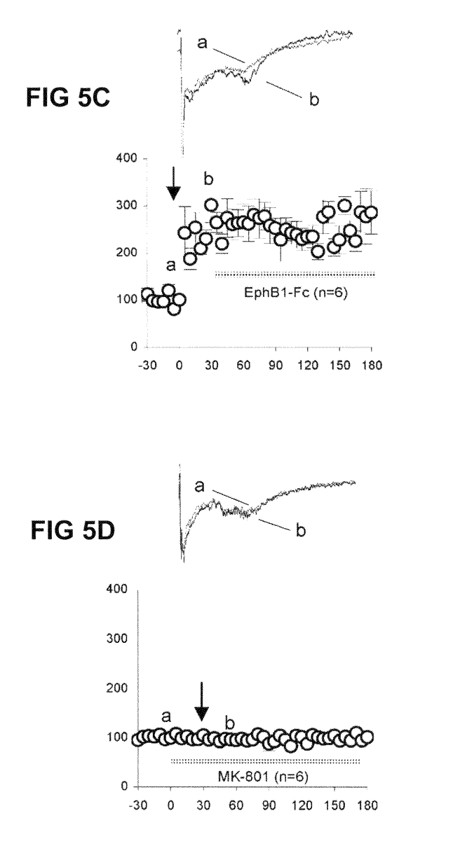

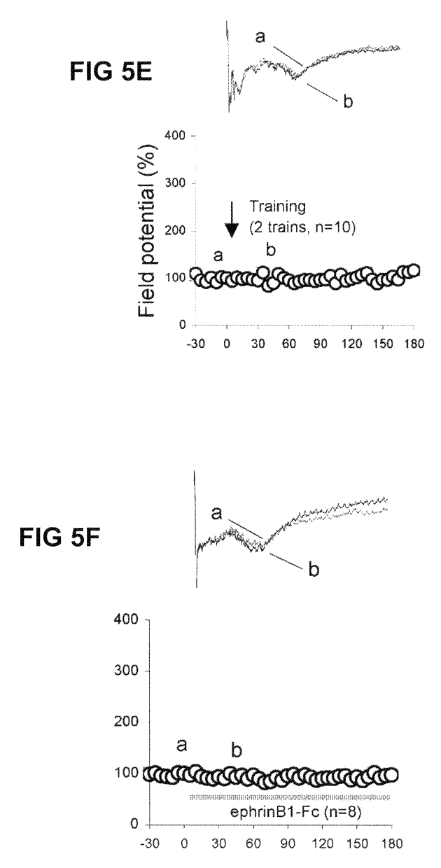

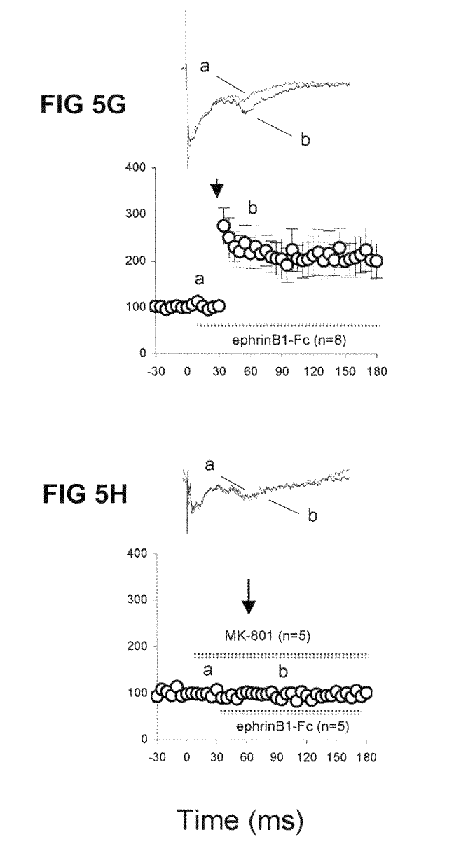

Long-term potentiation (LTP) of synapses between C-fibers and DH neurons may be associated with central sensitization contributing to painful consequences of inflammation and nerve injury (Liu and Sandkuhler, 1998; Matthews et al., 2006). The present study showed that 4 high-frequency trains of nerve shock produced LTP of these synapses in naïve animals (FIG. 5A) that was prevented by topical pre-treatment with the EphB receptor blocking reagent, EphB1-Fc (10 μg) (FIG. 5B), but was not affected by post-treatment with EphB1-Fc (FIG. 5C). The LTP induced by this protocol was also blocked by pre-treatment with an NMDA receptor antagonist, MK-801 (20 μg) (FIG. 5D), but was not interrupted by post-treatment with MK-801 (20 μg, n=4, data not shown). Further, the present study showed that the EphB receptor activator, ephrinB1-Fc, lowered the threshold for inducing LTP. Neither ephrinB1-Fc alone (5 μg, 3-5 hr) nor only 2 trains of nerve shock induced LTP under normal conditions (FIGS. 5E and 5F). However, pre-treatment with ephrinB1-Fc at the same dose 30 min prior to training permitted 2 trains to induce LTP (FIG. 5G). This LTP induced by the combination of ephrinB1-Fc and 2 tetanic trains was also blocked by pre-treatment with MK-801 (n=5) (FIG. 5H). EphrinB1-Fc and EphB1-Fc did not change the baselines of the field potentials. These findings show that activation of EphB receptors can regulate NMDA receptor-dependent synaptic plasticity within pain pathways in the DH.

Expression of EphrinB and EphB Receptors Proteins Increases in DRG and DH after Nerve Injury

Given that activation of EphB receptors was shown to be necessary and sufficient for behavioral and neuronal alterations associated with neuropathic pain, the present study further investigated whether ephrinB and EphB receptors were upregulated after nerve injury. Western blot procedures were used to measure possible changes in ephrinB and EphB receptor levels in the DRG and DH. Both ephrinB and EphB receptor proteins were expressed in both the spinal cord and DRG at relatively low levels in sham-operated rats, but CCI caused rapid upregulation of ephrinB and EphB receptor proteins at both sites (FIGS. 6A-6F). The expression of ephrinB and EphB receptor proteins increased significantly in the spinal cord ipsilateral to the injury within 1 day after nerve injury, remained at high levels for 1-14 days, and remained significantly elevated for at least 28 days after CCI. The expression of ephrinB in DRG also increased, but exhibited somewhat different patterns from that in the spinal cord, i.e., ephrinB significantly increased by day 1 but then gradually reached peak values at day 7-14 and only declined slightly by the 28^{th} day (FIGS. 6A and 6C). EphB receptor expression in the DRG showed similar patterns to that in the spinal cord, but the peak values tended to be less in the DRG (FIGS. 6D and 6F). The expression of ephrinB and EphB receptors also increased in the DRG and spinal cord contralateral to the injury, but these were always significantly less than ipsilateral increases (FIGS. 6B, 6C, 6E and 6F).

Sites of Increased Expression of EphrinB and EphB Receptors in DRG and DH after Nerve Injury

Illustrated in FIGS. 7A-7F is immunohistochemical labeling, which shows that 7 days after nerve injury, ephrinB labeling was greater in DRG neurons of CCI rats than of sham-operated rats, and was located largely in the cytoplasm (CCI in FIG. 7A), while EphB receptor labeling was also increased but was located near the cell membrane (CCI in FIG. 7B). The degree of the increase of EphB receptor expression after CCI appeared to be less than that of ephrinB, which was consistent with the results from Western blot analysis (compare FIGS. 6C 6F). In the spinal DH, the increased expression of ephrinB and EphB receptor was distributed throughout the DH. Interestingly, in CCI-treated but not sham-treated animals almost all of the IB4 positive nociceptive terminals in the DH contained ephrinB (FIG. 7E). In contrast, EphB receptor did not seem to be expressed in the IB4 positive nociceptive terminals (FIG. 7F).

In addition, labeling of neurons with the anti-neuronal nuclear protein (NeuN, Chemicon) showed that both ephrinB and EphB receptor were expressed in neurons in the DRG and DH, and this neuronal expression was increased by prior CCI (FIGS. 8A-8D). These data strongly suggest that nerve injury increases levels of ephrinB and EphB receptors in nociceptive IB4-positive DRG neurons and in DH neurons, but does not exclude the possibility of increased expression in other cell types as well.

### Discussion

The present study provides the first demonstration that ephrinB-EphB signaling contributes to neuropathic pain. Peripheral nerve injury, which led to neuropathic pain, triggered an upregulation of ephrinB and EphB receptors in nociceptive dorsal root ganglion (DRG) and dorsal horn (DH) neurons. Intrathecal administration of blocking reagents for EphB receptors prevented the induction of and transiently inhibited the expression of thermal hyperalgesia and mechanical allodynia produced by peripheral nerve injury, and blocked hyperexcitability of nociceptive DRG neurons, sensitization of spinal DH neurons, and long-term potentiation (LTP) of synapses between C fibers and DH neurons. Activators of EphB receptors induced a prolonged thermal hyperalgesia in naïve animals, enhanced hyperexcitability of nociceptive DRG neurons, and promoted LTP of synapses between DRG neurons and DH neurons.

These results are consistent with both pre- and post-synaptic actions of EphB receptors in the context of nerve injury. The hyperexcitability of DRG neurons induced by EphB receptor activation, and the blockade of CCI-induced DRG neuron hyperexcitability by blocking reagents for EphB receptors suggests widespread effects of these receptors on the excitability of pre-synaptic primary sensory neurons and represents the first demonstration that ephrin-Eph receptor signaling can regulate excitability in any neuron. The present findings that LTP of synapses between DRG and DH neurons requires ephrinB-EphB receptor signaling, and that a subthreshold LTP procedure becomes suprathreshold in the presence of ephrinB1-Fc indicates that ephrinB-EphB receptor signaling makes important contributions to synaptic plasticity in pain pathways. Accumulation of ephrinB and EphB receptors after chronic constriction injury (CCI) in both pre-synaptic sensory neurons and post-synaptic DH neurons suggests that ephrinB-EphB signaling may be important on both sides of the synapse, and leaves open the possibility that both forward and reverse ephrinB-EphB signaling occur pre- and post-synaptically.

Upregulation of ephrinB and EphB receptors were detected following nerve injury, although available blocking reagents did not identify the specific ephrinB and EphB receptors that were activated. However, the specificity of the antibodies used in the present study for detecting ephrinB1 and EphB1 suggested that the specific proteins of ephrinB1 and EphB1 receptors were upregulated after nerve injury.

A study of Battaglia et al. (2003) demonstrated that intrathecal administration of EphB receptor activators, ephrinB1-Fc or ephrinB2-Fc, could decrease threshold of LTP in DH neurons and produce thermal hypersensitivity in naïve rats. However, no mechanical allodynia was induced after intrathecal injection of EphB receptor activators in both Battaglia et al. (2003) and the present study. This might be related to a lack of effect of these activators on nociceptive DRG neurons from naïve rats. In the absence of injury, ephrinB may be involved more in input from thermal nociceptors than from low-threshold mechanoreceptors. However, after nerve injury, ephrinB-EphB signaling becomes important for both thermal hyperalgesia and mechanical allodynia.

Previous studies have shown that EphB receptors can regulate development of normal function and plasticity at glutamatergic synapses in the hippocampus (Dalva et al., 2000; Grunwald et al., 2001; Takasu et al., 2002) and in adult spinal cord between axons of DRG neurons and DH neurons (Battaglia et al., 2003) by interacting with N-methyl d-aspartate (NMDA) receptors (Ghosh, 2002; Calo et al., 2006). The present study indicate that ephrinB activated post-synaptic EphB receptors on DH neurons, which then promoted LTP and behavioral hyperalgesia by interacting with NMDA receptors. In particular, ephrinB1-Fc-induced thermal hyperalgesia was prevented by pretreatment of NMDA receptor antagonist MK-801 (FIGS. 2A-2B); LTP induced by subthreshold tetanic stimulation plus ephrinB1-Fc was prevented by pretreatment with MK-801, and the LTP prevented by pretreatment with EphB1-Fc was also sensitive to MK-801 (FIGS. 5A-5H).

Among ephrinB and EphB receptors, EphB2 was previously shown to be associated with as well as induce clustering of NMDA receptors (Dalva et al., 2000), and ephrinB1 was previously shown to enhance NMDA receptor-mediated Ca²⁺ influx and potentiate phosphorylation of cAMP-response element-binding protein (CREB) (a transcription factor) (Grundwald et al., 2001; Takasu et al., 2002). EphB2 receptor activation was found to be able to recruit and activate Src-family kinases, which then phosphorylate the NMDA receptor (Grundwald et al., 2001; Takasu et al., 2002). Interactions of EphB receptors with other signals may also contribute to post-synaptic effects and behavioral pain after nerve injury. The previous study also showed that expression of EphB2 was increased when the cells exposed to forskolin, and a surge of cAMP could trigger transcriptional activity to augment expression of EphB2 receptor genes (Jassen et al., 2006). Interestingly, cAMP-PKA activity was found to contribute to sensory neuron hyperexcitability and hyperalgesia after DRG compression (Song et al., 2006; Zheng et al., 2007). It remains to be investigated whether interactions between ephrinB-EphB receptors and the cAMP-PKA pathway are important for neuropathic pain.

Furthermore, a previous study reported that ephrinB3 was expressed in myelinated oligodendrocytes and inhibited neurite outgrowth in vitro to an extent similar to myelin-associated glycoprotein (Benson et al., 2005), and suggested that ephrin-Eph signaling may also be involved in inhibitory effects of myelin during neural regeneration (Benson et al., 2005). It remains to be investigated whether blocking ephrin-Eph signaling after nerve injury could have dual therapeutic benefits of enhancing axonal regeneration and relieving symptoms of neuropathic pain.

While the invention has been shown in only a few of its forms, it should be apparent to those skilled in the art that it is not so limited but susceptible to various changes without departing from the scope of the invention.

What is claimed is:

1. A method for treating a chronic pain, comprising:
administering to an individual in need of such treatment with a pharmaceutically effective amount of a blocking reagent for ephrinB-EphB signaling.

2. The method of claim 1, wherein said chronic pain comprises a neuropathic pain.

3. The method of claim 2, wherein said chronic pain further comprises an inflammatory pain.

4. The method of claim 2, wherein said neuropathic pain is caused by peripheral nerve injury or chronic compression of dorsal root ganglion, spinal nerves and/or dorsal roots.

5. The method of claim 1, wherein said blocking reagent is an EphB receptor blocker.

6. The method of claim 5, wherein said EphB receptor blocker comprises EphB1-Fc.

7. The method of claim 5, wherein said EphB receptor blocker comprises EphB2-Fc.

8. The method of claim 1, wherein said blocking reagent is administered in an amount of from about 0.5 μg to about 50 μg.

9. The method of claim 8, wherein said blocking reagent is administered in an amount of from about 5 μg to about 10 μg.

10. The method of claim 1, wherein said blocking reagent is administrated intrathecally.

11. The method of claim 1, wherein said individual is a mammal.

12. The method of claim 11, wherein said mammal is human.
